(19) United States
(12) Patent Application Publication
(10) Pub. No.: US 2006/0184132 A1
(43) Pub. Date: Aug. 17, 2006

(54) Hypodermic syringe

(76) Inventor: Robert Baird Watson, Victoria (AU)

Correspondence Address: KUDIRKA & JOBSE, LLP, ONE STATE STREET, SUITE 800, BOSTON, MA, 02109, US

(21) Appl. No.: 10/550,823

(22) PCT Filed: Mar. 24, 2004
(86) PCT No.: PCT/AU04/00364

§ 371 Date: Sep. 23, 2005

(30) Foreign Application Priority Data
Mar. 25, 2003 (AU) 2003901382

(51) Int. Cl. A61M 5/32

(52) U.S. Cl. 604/197

(57) ABSTRACT

A syringe (1), including: a syringe casing (2), and a syringe body (3) within the casing (2) and defining a chamber (4) for holding a charge of liquid. The syringe body (3) is controllably moveable relative to the casing. A hollow needle (13) is connected to the syringe body (3) for movement therewith and extends from the casing (2) for use of the syringe (1). A plunger (14) is reciprocally moveable within the body (3) for drawing liquid into the body chamber (4) and/or ejecting liquid from the body chamber (4) through the needle (13). Control means (22) enables the syringe (1) to draw and/or eject a charge of liquid through the needle, whereupon movement of the syringe body (3) and needle (13) relative to the casing (2) disables the syringe (1).

### BACKGROUND OF THE INVENTION

1. Field of the Invention

This invention relates to a non-reusable hypodermic syringe. The syringe is applicable for the administration of liquid medicines, pharmaceuticals, drugs and other solutions into human bodies, and it will be convenient to hereinafter disclose the invention in relation to that exemplary application. However, it is to be appreciated that the invention is not limited to that application. In that regard, the syringe may also be applicable to taking blood samples or other liquid from human bodies for analysis.

2. Description of the Prior Art

The following discussion of the background to the invention is intended to facilitate an understanding of the present invention. However, it should be appreciated that the discussion is not an acknowledgement or admission that any of the material referred to was published, known or part of the common general knowledge in Australia as at the priority date of the application.

Reuse of syringes is a significant cause of infection in human beings. Moreover, diseases can be relatively easily transmitted between humans during shared use of syringes. Used syringes are also a source of injury, and possible disease transmission, when they are inappropriately discarded with the hollow needle exposed following use.

In an effort to reduce these health problems, syringes have been developed which can be rendered inoperable and relatively harmless following a single use. In one such arrangement, the needle can be retracted into the syringe tubular body by the plunger following use so that the body then protectively houses the needle. A facility is provided which then prevents the needle being re-extended from the body, thereby rendering the syringe useless and harmless.

One such facility provides locking of the plunger stem and/or breaking off the stem, in order to prevent further movement of the plunger. Another facility involves axially offsetting the needle from a hole in the forward end of the body through which the needle initially extends from the body. In this way, when the needle is retracted into the syringe body, the needle and hole axially misalign thereby preventing the needle from passing back through the hole.

These syringes can be effective in preventing their multiple use, and also achieving safe disposal. However, one disadvantage with many of those syringes is that they are not tamper proof. They require positive action on the part of the user in order to render them inoperative and safe following a single use. The absence of that action, or misuse, will enable the syringes to be repeatedly used. Moreover, careless disposal will leave the hollow needle exposed with the potential for injury and illness.

Australian patent 731159 and patent application 2001252019 disclose other retractable, single use syringes. Those syringes can be effective in preventing multiple use and also enabling safe disposal. However, the complexity of the means of the facility disabling the syringe leads to manufacturing costs which are high relative to other syringes.

### SUMMARY OF THE INVENTION

It is an object of the present invention to provide a syringe which is less susceptible to misuse and abuse.

It is an object of the present invention to provide a syringe which effectively prevents re-use.

It is an object of the present invention to provide a syringe which can be automatically rendered harmless following a single use.

It is a still further object of the present invention to provide a syringe of simpler construction and lower manufacturing cost whilst retaining effectiveness against misuse and abuse.

In broad terms, the present invention provides a syringe including:

a syringe casing;

a syringe body within the casing and defining a chamber for holding a charge of liquid, the syringe body being controllably moveable relative to the casing;

a hollow needle connected to the syringe body for movement therewith and extending from the casing for use of the syringe;

a plunger reciprocally moveable within the body for drawing liquid into the body chamber and/or ejecting liquid from the body chamber through the needle; and,
control means enabling the syringe to draw and/or eject a charge of liquid through the needle, whereupon movement of the syringe body and needle relative to the casing disables the syringe.

Preferably, the syringe body is controllably movable relative to the casing from a position in which the syringe is enabled for drawing and/or ejecting the liquid charge, to a position in which the syringe is disabled preventing syringe use. The control means preferably effects controlled movement of the body from the enabled position to the disabled position. In one preferred form, the syringe body is controllably movable from the enabled position to a disabling position in which the syringe is positioned for disablement, and then from the disabling position to the disabled position. The control means effects controlled movement of the body through the disabling position, in this form.

The syringe casing and body are each elongate, and the syringe body is axially and rotatably slideable within the syringe casing. The syringe body preferably slides from the enabled position to the disabled position. Preferably, the syringe body is axially slidable into the disabled position, sliding of the syringe body into the disabled position retracting the needle into the casing to thereby disable the syringe.

Preferably, the control means includes at least one control member on the syringe casing, and at least one control member on the syringe body. Those control members on the syringe casing and body preferably inter-engage during syringe use to effect the controlled movement of the body. In one preferred form, the control members include at least one control cam and at least one cam follower, the cam and cam follower inter-engaging to effect the controlled movement of the body. In this form, the cam may be located on the body, and the cam follower located on the casing.

Preferably, the cam follower is a follower pin.

Preferably, the control cam is elongate and has at least one profiled camming surface extending therealong for operative engagement by the cam follower. In one preferred form, the control cam includes a camming groove or slot for receiving the cam follower pin therein. The cam follower pin progressive travels along the camming groove or slot causing relative movement between the syringe body and casing in response to the camming surface profile, in this form.

Preferably, the control cam includes camming stages spaced there along, each with a respective camming surface with which the cam follower successively travels over to cause indexed movement of the syringe body relative to the casing through each camming stage.

Preferably, the camming stages including one or more of:

a. a charge draw camming stage during which a liquid charge is drawn into the body chamber;

b. an air ejection camming stage during which air is ejected from the needle;

c. a blood draw camming stage during which blood is drawn from a vein into the needle; and,

d. a charge ejection camming stage during which a liquid charge is ejected from the body chamber,

Movement of the plunger during each camming stage preferably forces the follower into engagement with and to travel over the respective camming surface and thereby cause the syringe body to rotatably slide relative to the syringe casing. In one preferred form the camming stage are provided in the above order a, b, c and d along the control cam.

Preferably, the control cam includes detent stages adjacent the end of each of the camming stages for ending travel of the cam follower over the preceding camming stage and directing the cam follower toward the successive camming stage. Each detent stage preferably has a respective camming surface. Those camming surfaces are angled relative to the camming surfaces in the preceding camming stages so as to redirect travel of the cam follower and cause the indexed movement of the syringe body through each camming stage.

Preferably, the control cam includes a disabling camming stage during which the cam follower and cam disengage and the syringe body moves toward the disabled position. The camming groove or slot preferably has an open end through which the follower pin moves to exit from the camming groove or slot so as to release the syringe body for longitudinal sliding movement relative to the syringe casing to the disabled position and thereby retracting the needle into the casing.

Preferably, the syringe includes biasing means acting on the syringe body to slidably move the body when in the disabling position to the disabled position. In one preferred form, the biasing means includes a resilient biasing spring acting between the syringe casing and body, to bias the body along the casing into the disabled position.

Preferably, the syringe body is moveable from an enabling position, in which the needle is retracted and housed within the casing, to the enabled position. The control cam preferably includes an enabling camming stage, movement of the syringe body to the enabled position causing the cam follower to engage the cam and travel over the camming surface of the enabling camming stage. Movement of the syringe body projects the needle from the casing for use of the syringe. Preferably, the camming groove or slot has an open end through which the follower pin moves to enter the camming groove or slot so as to engage the camming surface of the enabling camming stage.

In one arrangement, the syringe casing includes an access hole through which the needle extends during use of the syringe. That access hole is offset from the needle so that, when the needle is retracted into the casing into the disabled position, the retracted needle misaligns with the access hole so as to prevent re-extension of the needle from the casing.

In another arrangement, locking means are provided for acting on the body to prevent subsequent movement thereof relative to the casing when in the disabled position.

### DESCRIPTION OF THE DRAWINGS

The following description refers to a preferred embodiment of the syringe of the present invention. To facilitate an understanding of the invention, reference is made in the description to the accompanying drawings where the syringe is illustrated in that preferred embodiment. It is to be understood that the syringe is not limited to the preferred embodiment as hereinafter described and as illustrated in the drawings.

In the drawings:

FIG. 1 is a perspective view, partially in section, of a hypodermic syringe according to a preferred embodiment of the present invention, showing the syringe in an enabling position;

FIG. 2 is a perspective view of the body of the syringe of FIG. 1;

FIG. 3 is a longitudinal cross sectional side view of the syringe of FIG. 1, showing the syringe moved into an enabled position;

FIG. 4 is a side view similar to FIG. 3 of a part of the syringe but showing the syringe in an enabled position and the chamber holding a charge of liquid;

FIG. 5 is a side view similar to FIG. 4 but showing the syringe in an enabled position and air having been ejected from the needle;

FIG. 6 is a side view similar to FIG. 5 but showing the syringe in an enabled position and blood having been drawn from a vein into the needle;

FIG. 7 is a side view similar to FIG. 6 but showing the syringe after ejection of the charge from the chamber and the syringe in a disabling position; and,

FIG. 8 is a perspective view, partially in section of the syringe of FIG. 1 but showing the syringe in a disabled position.

### DETAILED DESCRIPTION OF THE PREFERRED EMBODIMENT OF THE INVENTION

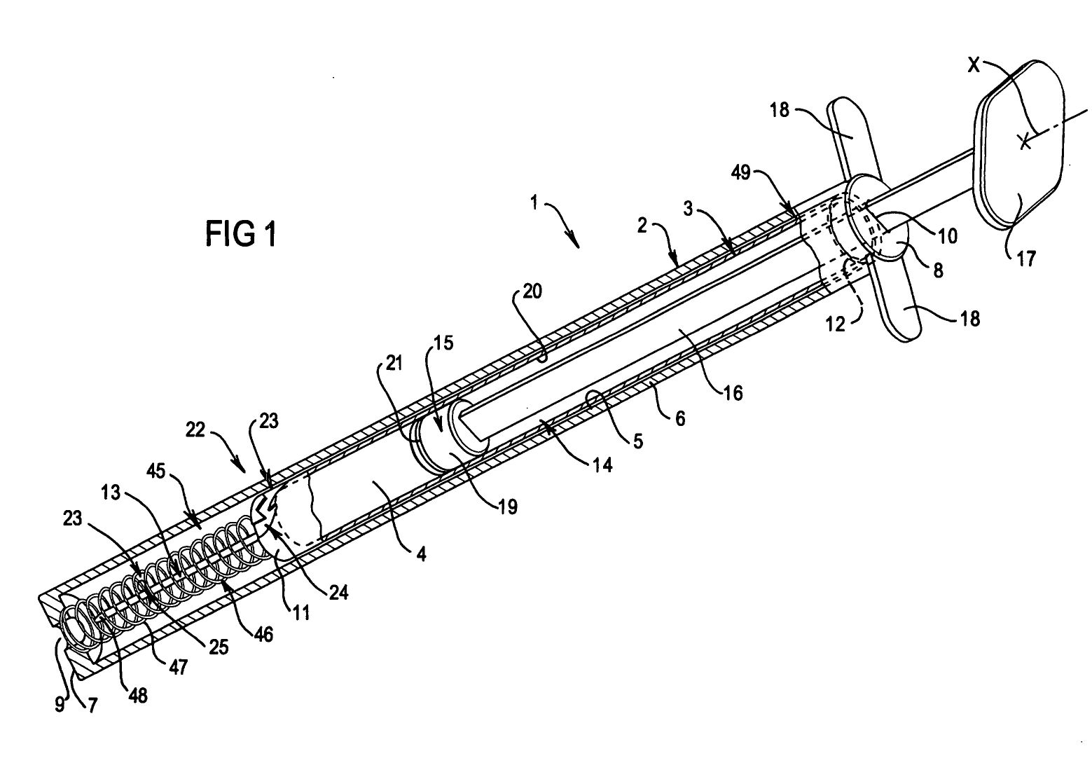

Referring initially to FIG. 1, there is generally shown a syringe 1 having an elongate syringe casing 2 and an elongate syringe body 3 located within the casing 2. The body 3 defines a chamber 4 for holding a charge of liquid. In the exemplary application the liquid charge is a medicine, pharmaceutical drug or other solution for administration into a human body, or a blood sample taken from the body.

The casing 2 is cylindrical in cross-sectional shape with inner and outer surfaces 5, 6, respectively. The casing 2 also has opposed forward and rear ends 7, 8, respectively.

The syringe casing 2 is at least substantially closed at each end 7, 8. In particular, the casing 2 is sealed at each end except for access openings 9, 10, the purposes of which will become more apparent hereinafter.

The syringe body 3 is also elongate with opposed forward and rear ends 11, 12, respectively. The body 3 extends along within the casing 2 for movement therealong. As shown, the casing 2 and body 3 are orientated with their forward ends 7, 11 in the same one direction and the rear ends 8, 12, in the same opposite directions.

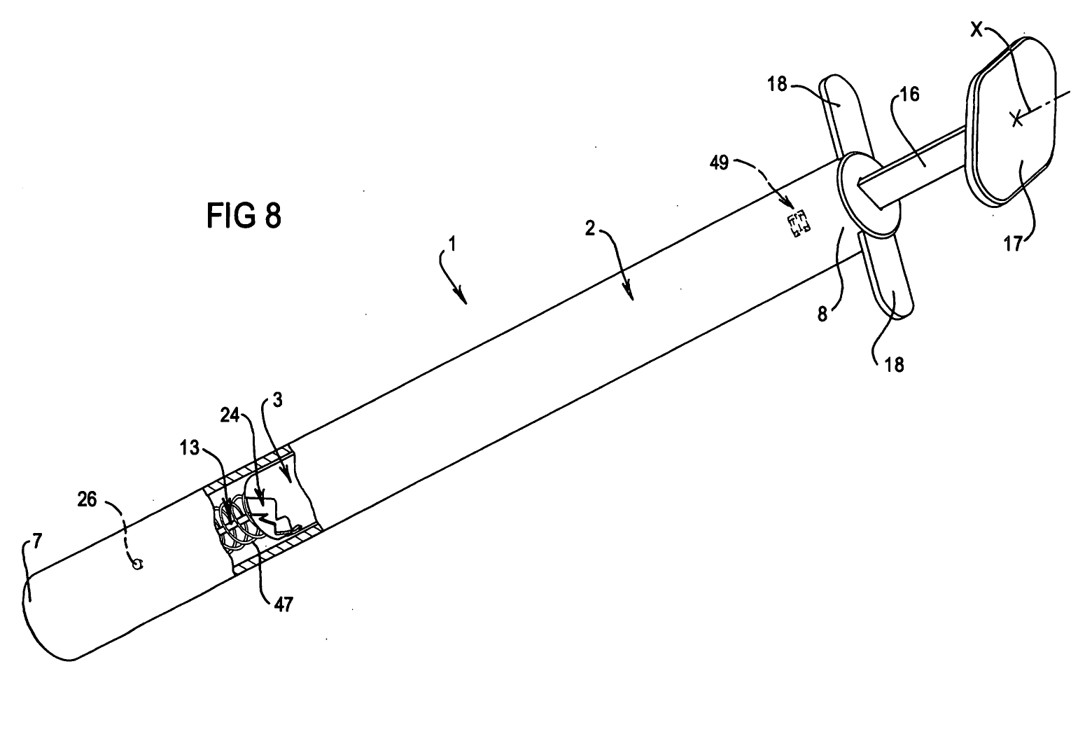

The syringe body 3 is controllably movable from a position in which the syringe is enabled to a position in which the syringe is disabled. In the enabled position, (as shown in FIGS. 4 to 6) the body 3 is positioned toward the forward end 7 of the casing 2, whilst in the disabled position (as shown in FIG. 8) the body 3 is positioned further away from the forward end 7 of the casing 2. In the enabled position the body 3 is positioned with its forward end 11 adjacent the forward end 7 of the casing 2. In the disabled position the body 3 is positioned with its forward end 11 spaced from the forward end 7 of the casing 2, and its rear end 12 may be adjacent the rear end 8 of the casing 2.

In moving from the enabled position to the disabled position, the body 3 moves through a disabling position. Thus, the body 3 is movable from the enabled position to the disabling position and then to the disabled position. In the disabling position (as shown in FIG. 7) the body 3 is positioned for disablement of the syringe 1.

The body 3 is tubular and axially slidable within the casing 2. The tubular body 3 is also rotatably slidable about its longitudinal axis X relative to the casing 2. As shown, the tubular body 3 is cylindrical in cross sectional shape and is a neat sliding fit within the tubular casing 2. The body 3 is generally rotatably slidable from the enabled position to the disabling position, and generally axially slidable from the disabling position to the disabled position.

The syringe 1 includes a hollow needle 13 connected to the forward end 11 of the syringe body 3 for movement therewith. The needle 13 projects forwardly from the syringe body 3 through the access hole 9 in the forward end 7 of the casing 2 in order to extend from the casing for use of the syringe.

The needle 13 is in communication with the chamber 4. In this way, the liquid charge can be drawn through the hollow needle 13 into the chamber 4 and subsequently ejected therefrom through the needle 13. Depending on the intended application of the syringe 1, the liquid might be, for example, an injectable solution drawn from a supply container and subsequently ejected/injected subcutaneously, or blood drawn subcutaneously from a body and subsequently ejected for analysis.

The tubular body 3 is closed at the forward end 11, and the needle 13 is fitted into that forward end 11 so as to permanently secure the needle 13 to the body 3 and place the needle 13 in communication with the chamber 4.

A plunger 14 is reciprocally movable within the syringe body 3 for drawing liquid into the body chamber 4 and ejecting liquid from the chamber 4 through the needle 13. The plunger 14 includes a piston 15 slidably movable within the body chamber 4, and an actuating stem 16 extending from the piston 15 and through the rear ends 12, 8 of the body 3 and casing 2. The stem 16 extends through the access hole 10 in the otherwise closed rear end 8 of the casing 2.

The stem 16 is non-circular in transverse cross section, and the access hole 10 through which the stem 16 extends is of the same cross sectional shape. In this way, the plunger 14 can be reciprocated within the syringe body 3 but is prevented from rotating relative thereto.

That stem 16 is manually actuable in order to move the piston 15 within the chamber 4 so as to draw and eject liquid through the needle. To facilitate manual actuation of the stem 16, a thumb rest 17 may be provided on an end of the stem 16, and one or more finger flanges 18 may be provided on the syringe casing 2 at or adjacent the rear end 8, in a manner well understood by those skilled in the relevant art.

The piston 15 has an outer surface 19 that is in liquid sealing engagement with an inner surface 20 of the tubular body. In this way, liquid can be drawn into and ejected from the chamber 4 without leaking past the piston 15. Sealing engagement may be achieved by forming the piston 15, or at least the outer surface 19 thereof, of sealing material, or by mounting one or more sealing members such as sealing rings 21 on the piston 15. The sealing material or member(s) may be composed of rubber.

Control means 22 is provided for controlling movement of the syringe body 3 from the enabled position to the disabled position. The control means 22 includes at least one control member 23 on the syringe casing 2, and at least one control member 23 on the syringe body 3, the control members 23 interengaging during syringe use to thereby effect the controlled movement of the body from the enabled position to the disabling position and then to the disabled position. The engagement between the control members 23 also maintains the casing 2 and body 3 in relative longitudinal disposition while the syringe 1 is enabled (i.e. while the body 3 rotates from the enabled position to the disabling position), but is releasable under the controlled movement of the body for disabling the syringe 1. That release allows the body 3 to move axially from the disabling to the disabled position and thereby retract the hollow needle 13 into the casing 2 so as to disable the syringe 1.

The control members 23 include at least one control cam 24 and at least one cam follower 25, the cam 24 and follower 25 interengaging to effect the controlled movement.

The or each cam 24 is located on the body 3, and the or each cam follower 25 is located on the casing 2, although it will be appreciated that these locations may be reversed.

As shown in this embodiment, a single control cam follower 25 is provided, that follower 25 being located on an inner surface 5 of the casing 2 and extending generally inwardly therefrom. The cam follower 25 is located adjacent the forward end 7 of the casing 2.

The cam follower 25 includes a follower projection. That projection is fixed to the inner surface 5 of the casing 2 and projects inwardly therefrom. In one preferred form, the projection is a follower pin 26.

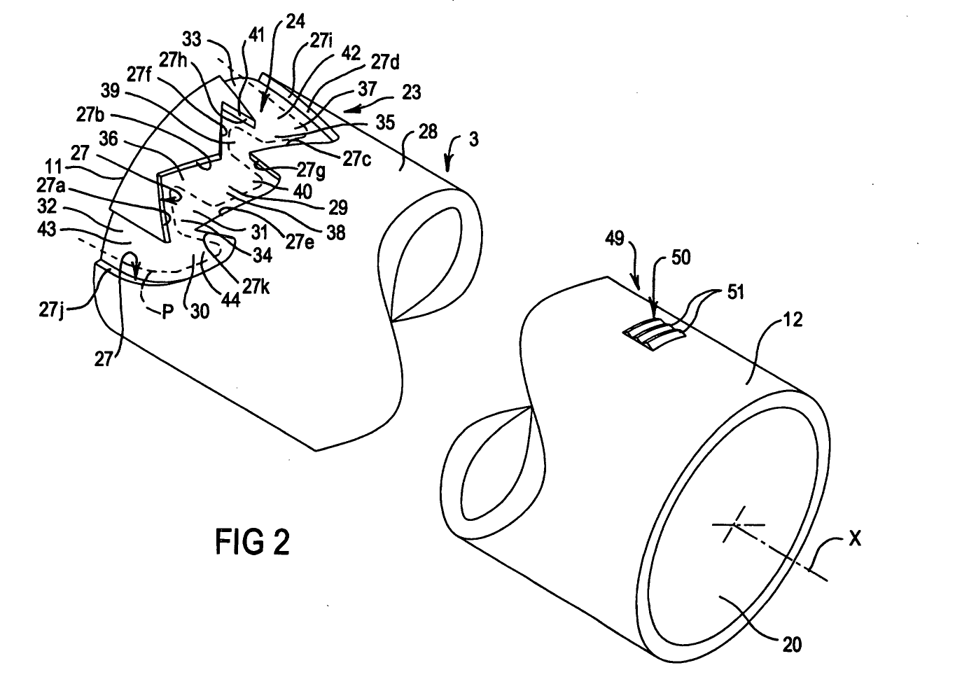

As shown in this embodiment, a single control cam 24 is provided. That cam 24 is shown in greater detail in FIG. 2, and has at least one profiled camming surface 27 for operative engagement by the single cam follower 25. The control cam 24 is provided at an outer surface 28 of the body 3, in facing relation to the cam follower 25 during engagement therebetween. To that end, the control cam 24 is located adjacent the forward end 11 of the body 3.

The control cam 24 includes a camming groove or slot 29 for receiving the cam follower pin 26 therein. Thus, the camming groove or slot 29 has an open top 30, and opposed side faces, one or both of which provides camming surface(s) 27. The control cam 24 is set into the wall of the body 3, with the open top 30 at the outer surface 28. The camming groove 29 (as shown) has a closed bottom 31, whilst the camming slot (not shown) has an open bottom. With this arrangement, during engagement, the follower pin 26 extends through the open top 30 into the camming groove or slot 29, and contacts the camming surfaces 27 in order to apply a force to the body 3 causing controlled movement of the body 3 from the enabled position to the disabled position.

The camming groove or slot 29 is of a discrete length with opposite ends 32, 33. At least one end 33 is open, so as to allow the follower 25 and cam 24 to disengage for disablement of the syringe 1. Specifically, as shown, end 33 is open to allow the follower pin 26 to exit from the camming groove or slot 29 and so disengage from one another.

As shown, both ends 32, 33 of the camming groove or slot 29 are open so that the pin 26 can respectively enter and exit the groove or slot 29 for the controlled movement of the syringe body 3. Thus, the pin 26 enters the camming groove or slot 29 at entry end 32 and progressively travels therealong causing relative movement between the body 3 and casing 2, in response to the camming surface 27 profile, to move the body 3 from the enabled position to the disabling position, before exiting from the exit end 33 so as to disable the syringe 1. Travel of the pin 26 along the camming groove or slot is on path P in one direction from entry end 32 to exit end 33.

The open ends 32, 33 of the groove or slot 29 open through the forward end 11 of the body 3.

The cam 24 has stages spaced therealong, each with a respective camming surface 27 and with which the follower 25 successively engages for indexed movement of the syringe body 3 relative to the casing 2. Those stages, include one in which the follower 25 travels during initial drawing of a liquid charge into the body chamber 4 (the charge draw camming stage 34), and another in which the follower 25 travels during ejection of the liquid charge from the chamber 4 (the charge ejection camming stage 35).

In one preferred form, the charge draw camming stage 34 prevents substantial longitudinal movement of the syringe body 3 relative to the casing 2, but permits rotation of the body 3 so that the follower 25 travels relatively toward the charge ejection camming stage 25. During that travel, the plunger 14 is able to be withdrawn relative to the body 3, so as to draw a charge of liquid into the chamber 4. That travel occurs and is completed during initial drawing movement of the plunger 14, as shown in FIG. 4 of the drawings.

To achieve that relative movement of the body 3 and casing 2, a camming surface 27a in the charge draw camming stage 34 extends at an angle to the longitudinal axis X of the syringe 1. In this way, engagement with the follower 25 forces the body 3 to rotate about the axis X.

In order to limit relative travel of the cam 24 and cam follower 25 to that camming stage 34 until the drawing movement of the plunger 14 is completed, the control cam 24 also includes a detent stage 36 adjacent the end of the charge draw camming stage 34. The detent stage 36 temporarily halts continued travel of the cam follower 25 along the cam 24. The cam follower 25 abuts camming surface 27b in the detent stage 36 to prevent that further travel.

The detent stage 36 is provided by a reverse angling of the camming surface 27b, relative to the camming surface 27a in the charge draw camming stage 34. In this way, the camming surfaces 27a, 27b define an included angle into which the travelling cam follower 25 is received and temporarily nested so as to impede further relative travel between the control cam 24 and cam follower 25.

The charge ejection camming stage 35 prevents substantial longitudinal movement of the syringe body 3 relative to the casing 2, but permits rotation of the body 3 so that the cam follower 25 continues to travel toward the exit end 33 of the control cam 24. During this travel, the plunger 14 is able to eject the charge of liquid from the chamber 4. That travel occurs, and is completed, during initial ejecting movement of the plunger. Completion of the travel positions the body 3 in the disabling position as shown in FIG. 7.

In order to achieve that relative movement, the camming surface 27c in the charge ejection camming stage 35 extends at an angle to the longitudinal axis X of the syringe 1. In this way, engagement with the cam follower 25 forces the body 3 to rotate about the axis X. The camming surface 27c in this stage 35 is on the opposite side face of the camming groove or slot 29 to the side face providing the camming surface 27a in the charge control camming stage 34, as shown in FIG. 2.

In order to limit relative travel of the cam 24 and cam follower 25, to that camming stage 35, until the charge ejection is completed, the control cam 24 includes a further detent stage 37 adjacent the end of the charge ejection camming stage 35. The further detent stage 37 temporarily halts continued travel of the cam follower 25 along the cam 24. The cam follower 25 abuts camming surface 27d in the detent stage 37 to prevent the further movement.

The further detent stage 37 is provided by reverse angling of the camming surface 27d. The camming surface 27d in the further detent stage 37 extends at least substantially parallel to the longitudinal axis X of the syringe 1. In this way, the camming surfaces 27c, 27d define an included angle into which the travelling cam follower 25 is received and temporarily nested to inhibit further relative travel between the cam 24 and cam follower 25.

In one arrangement (not shown) the charge draw camming stage 34 and charge ejection camming stage 35 are arranged adjacent one another, interposed by the detent stage 36. In this arrangement, successive movements of the plunger 14 draws a charge of liquid into the body chamber 4 and then ejects that charge through the needle 13.

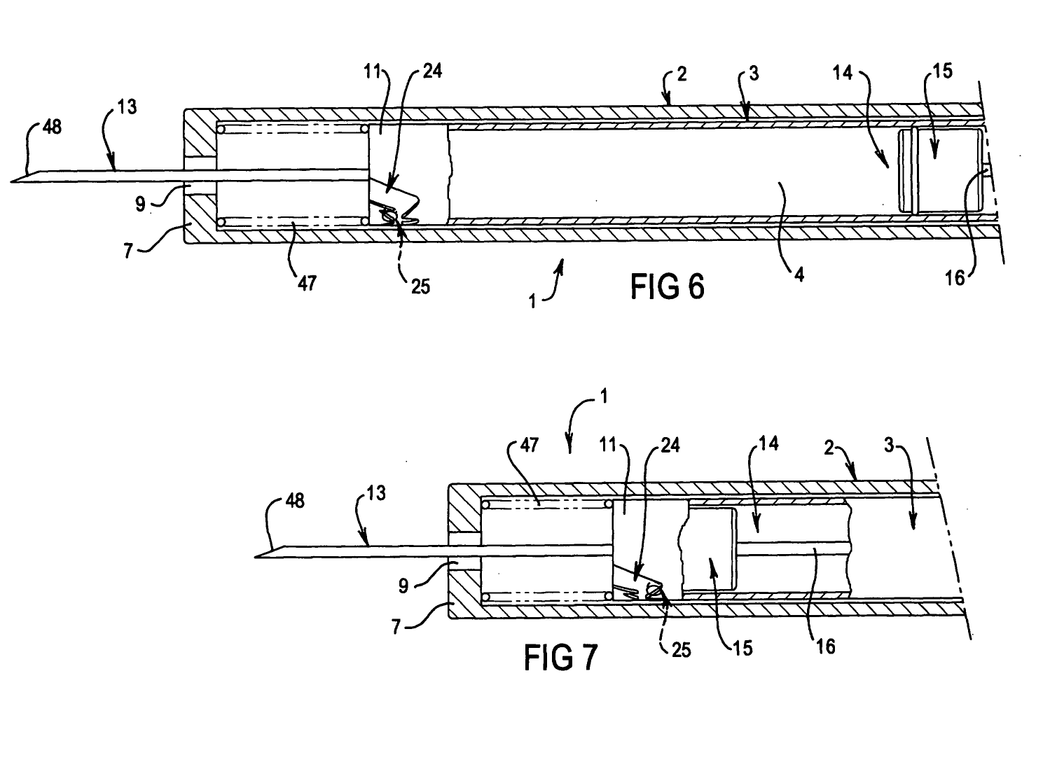

In an alternative arrangement (as shown with particular reference to FIGS. 5 and 6), however, the control means 22 can permit one or more auxiliary movements of the plunger 14 between charge drawing and ejection. By way of example, those movements may be to eject air from the needle 13 following charge drawing, and to draw blood from a vein into the needle 13 prior to charge ejection into that vein. In one preferred arrangement (as shown), both movements can be provided by the control means 22.

To that end, in this alternative arrangement, the control cam 24 includes an air ejection camming stage 38 and a blood draw camming stage 39, respectively.

In this form, the air ejection and blood draw camming stages 38, 39 are arranged successively between the charge draw and ejection draw camming stages 34, 35. In particular, the air ejection camming stage 38 succeeds the detent stage 36 adjacent the end of the charge draw camming stage 34, and the blood draw camming stage 39 precedes the charge ejection camming stage 35.

Each of the air ejection and blood draw camming stages 38, 39 prevent substantial longitudinal movement of the syringe body 3 relative to the casing 2, but permit rotation of the body 3 as with the charge draw and ejection camming stages 34, 35. During those movements, the plunger 14 is able to eject air from the needle 13 and draw blood from the vein into the needle 13. Again, those movements can occur and be completed during initial movement of the plunger 14, as shown in FIGS. 5 and 6 of the drawings, respectively.

In order to achieve the relative movement of the body 3 and casing 2, the camming surfaces 27e, 27f of the air ejection and blood draw camming stages 38, 39 extend at angles to the longitudinal axis X of the syringe 1. In this way, engagement with the cam follower 25 forces the body 3 to rotate about the axis X.

In order to limit relative movement of the cam 24 and cam follower 25, to the respective camming stages 38, 39 until air ejection and blood drawing is respectively completed, the control cam 24 includes additional detent stages 40, 41 adjacent the ends of the air ejection and blood draw camming stages 38, 39. Those detent stages 40, 41 are of the same general configuration as the detent stages 36, 37 adjacent the ends of the charge draw and ejection camming stages 34, 35, and have respective camming surfaces 27g and 27h.

The control cam 24 further includes a disabling camming stage 42. That stage 42 succeeds the charge ejection camming stage 35. In particular, the disabling camming stage 42 extends from the further detent stage 37 adjacent the end of the charge ejection camming stage 35 to the exit end 33 of the camming groove or slot 29. Travel of the cam follower 25 along that disabling camming stage 42 causes the body 3 to move from the disabling position to the disabled position as shown by the sequence of FIGS. 7 and 8 of the drawings.

The disabling camming stage 42 permits longitudinal movement of the body 3 relative to the casing 2 and retraction of the needle 13 into the casing 2. To that end, the camming surface 27i of disabling camming stage 42 can extend at least generally longitudinally of the syringe 1, although alternatively can also be angled relative to the longitudinal axis X of the syringe 1. In any event, the cam follower 25 is caused to exit from the camming groove or slot 29, so as to release the body 3 for movement longitudinally relative to the casing 2.

In one arrangement (not shown), the syringe 1 is initially supplied in the enabled position, i.e. with the body 3 positioned toward the forward end 7 of the casing 2. In that arrangement, the needle 13 will project from the casing 2.

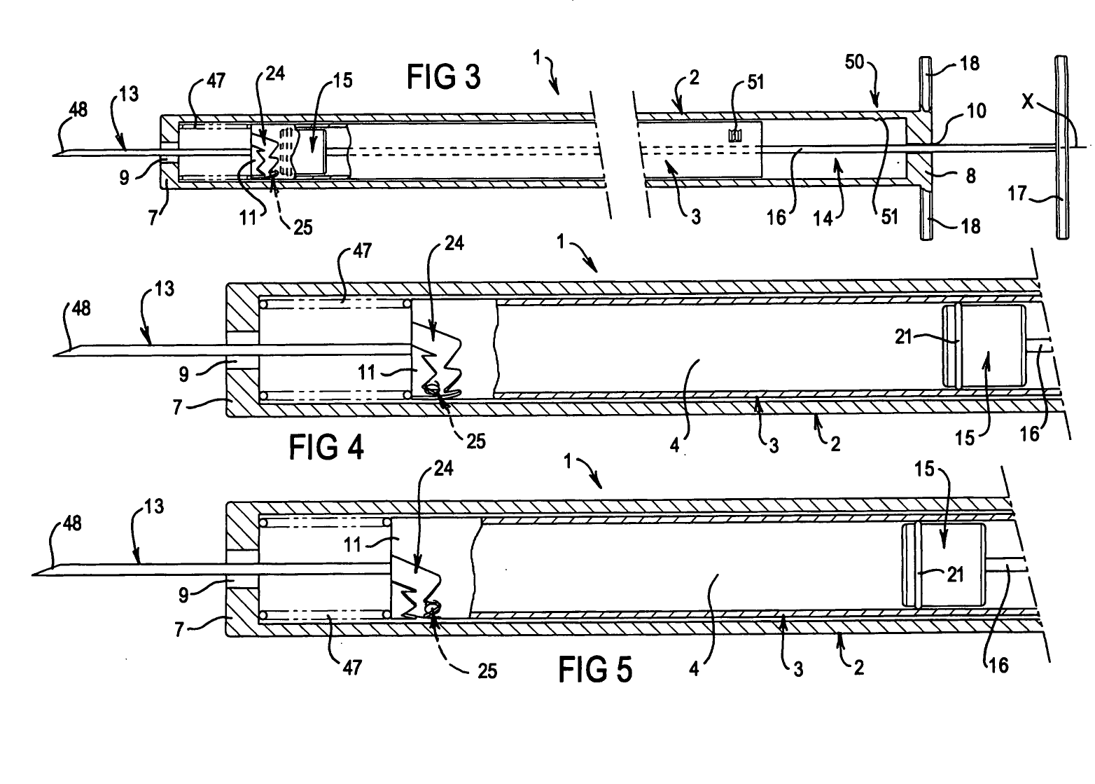

However, in an alternative arrangement (as shown in FIG. 1 of the drawings) the syringe 1 is supplied with the body 3 positioned away from the forward end 7 of the casing 2 so that the needle 13 is retracted and housed within the casing 2. That would protect the needle 13 from damage or misuse. In that enabling position, the body 3 would need to be moved toward the forward end 7 of the casing 2 so that the syringe 1 is in the enabled position (FIGS. 3 to 6 of the drawings).

In this alternative arrangement, the control cam 24 further includes an enabling camming stage 43. That camming stage 43 precedes the charge draw camming stage 34, extending from the entry end 32 of the camming groove or slot 29 toward the charge control camming stage 34.

The entry end 32 of the control cam 24 is longitudinally aligned with the cam follower 25 when the body 3 is in the enabling position and before the syringe 1 is enabled. In this way, longitudinal movement of the body 3 toward the forward end 7 of the casing 2 for enablement causes the cam follower 25 to enter and move along the enabling camming stage 43. Movement of the body 3 relative to the casing 2 is caused by applying a force to the plunger stem 16 in a forward direction. The frictional engagement between the plunger piston 15 and inner surface 20 of the syringe body 3, as well as contact between the plunger piston 15 and forward end 11 of the body 3, ensures that the body 3 is moved.

The camming surface 27j of the enabling camming stage 43 of the cam 24 extends at an angle to the longitudinal axis X of the syringe 1. In this way, engagement with the cam follower 25 forces the body 3 to rotate about the axis X. As a result of the relative movement between the cam 24 and cam follower 25, the cam follower 25 is moved out of alignment with the entry end 32 of the camming groove or slot 29, and toward the charge draw camming stage 34. This movement is shown by the sequence of FIGS. 1 and 3 of the drawings.

In the enabled position shown in FIG. 3 the plunger piston 15 is adjacent the forward end 11 of the body 3. In order to limit relative movement between the cam 24 and cam follower 25 in the enabling camming stage 43, until the plunger piston 15 is in that position, the control cam 24 includes another detent stage 44 adjacent the end of the enabling camming stage 43. Thus, that other detent stage 44 is positioned between the enabling stage 43 and charge control camming stage 34. That other detent stage 44 has camming surface 27k, and the same general configuration and function as the previously described detent stages 36, 37, 40, 41.

As shown, the camming groove or slot 29 is of a generally zig zag configuration. With this configuration, the multiple stages of the control cam 24 are arranged with the detent stages 36, 37, 40, 41, 44 the camming stages 34, 35, 38, 39, 42, 43 arranged on opposite sides of apical points of the groove on slot 29.

The rearward longitudinal movement of the body 3, causing retraction of the needle 13 into the casing 2, can be achieved by reverse manual movement of the plunger 14. To that end, the plunger stem 16 may be gripped and pulled rearwardly by a user of the syringe 1.

However, as shown, that body movement from the disabling position to the disabled position can occur automatically. That is, the body 3 will automatically move rearward if a user simply removes the forward pushing force from the plunger stem 16 and fails to manually pull the stem 16 rearwardly after use of the syringe 1.

That body movement is caused by biasing means 45 acting on the body 3 to slidably move the body 3 rearwardly when in the disabling position. That biasing means 45 extends between the body 3 and casing 2.

The biasing means 45 includes a resilient biasing member 46. In one form (not shown), the biasing member 46 is a resilient strip or band extending between and connected to the body 3 and casing 2. That strip or band is resiliently stretched when the body 3 is in its disabling position and acts to pull the body 3 rearwardly into its disabled position upon release of the plunger 14 following syringe use. The strip or band can be connected to the body 3 and casing 2 at or adjacent the rear ends 8, 12 thereof.

In another form (as shown), the biasing member 46 is a compression spring 47 positioned between the body 3 and casing 2 at the forward ends 7, 11 thereof. That spring 47 is compressed when the body 3 is in its enabled position and while the body 3 is rotated into its disabling position. However in that disabling position, the cam 24 and cam follower 25 no longer constrain the body 3 against longitudinal movement and so the compression spring 47 acts to bias the body 3 along the casing 2 into its disabled position. As shown, the compression spring 47 is a helically coiled spring.

It should be appreciated that the biasing member 46 will also act during use of the syringe 1 to bias the body 3 toward the rear end 8 of the casing 2. That bias will be overcome by a manual force applied periodically to the plunger 14, such as during liquid charge drawing, tending to bias the body 3 toward the forward end 7 of the casing 2. The resultant of those axially biasing forces will cause engagement between the cam follower 25 and camming surfaces 27a to k with which the follower 25 is successively, axially aligned. Those forces will also cause the relative travel between the interengaging follower 25 and cam 24 in the camming stages 34, 35, 38, 39, 42, 43, in turn effecting indexed movement of the body 3 relative to the casing 2. That interengagement will continue until the follower 25 is aligned with the exit end 33 of the camming groove or slot 29, whereupon the follower 25 is free to disengage from the groove or slot 29 under bias of the biasing member 46 so that the body 3 moves to the disabled position.

The retracted needle 13 may be prevented from being re-extended out of the casing 2. Preventing re-extension of the needle 13 minimises the prospect that the syringe 1 could cause injury such as through careless disposal.

Although not shown, re-extension of the needle 13 may be prevented by axially offsetting the needle 13 and access hole 9 in the casing forward end 7. The extent of offset is selected so that the needle 13 will extend through the hole 9 during use of the syringe 1, but once retracted into the casing 2, will misalign with that hole 9 and so be unable to pass back through the hole 9 upon application of a forward force to the plunger 14. Indeed, that force will cause a leading free end 48 of the needle 13 to press against and possibly embed into the casing forward end 7 thereby destroying the usefulness of the needle 13.

Alternatively (as shown), re-extension of the needle 13 may be prevented by locking means 49 acting on the body 3 to prevent subsequent forward movement when in the disabled position. That locking means 49 acts between the body 3 and casing 2.

The locking means 49 includes at least one locking element 50 on each of the body 3 and casing 2, the locking elements 50 interengaging when the body 3 moves into the disabled position, thereby preventing further movement of the body 3.

The locking elements 50 are locking teeth 51. Those locking teeth 51 are located at or adjacent the rear ends 8, 12 of the body 3 and casing 2. A single tooth 51 may be provided on each of the body 3 and casing 2, whilst as shown, a plurality of teeth 51 arranged in a ratchet formation, may be provided on at least one of the body 3 and casing 2.

The syringe of the present invention is confined to a single proper use. Reuse of the syringe is effectively prevented. Moreover, following that use, the syringe can be easily rendered harmless, either by the user or, in one form, automatically. As a consequence, the syringe is less likely to cause transmission of diseases through re-use or misuse, or injury.

Finally, it is to be understood that various alterations, modifications and/or additions may be made to the syringe without departing from the ambit of the present invention as defined in the claims appended hereto.

1. A syringe, including:
a syringe casing;
a syringe body within the casing and defining a chamber for holding a charge of liquid, the syringe body being controllably moveable relative to the casing;
a hollow needle connected to the syringe body for movement therewith and extending from the casing for use of the syringe;
a plunger reciprocally moveable within the body for drawing liquid into the body chamber and/or ejecting liquid from the body chamber through the needle; and,
control means enabling the syringe to draw and/or eject a charge of liquid through the needle, whereupon movement of the syringe body and needle relative to the casing disables the syringe.

2. A syringe as claimed in claim 1, wherein the syringe body is controllably movable relative to the casing from a position in which the syringe is enabled for drawing and/or ejecting the liquid charge, to a position in which the syringe is disabled preventing syringe use, and the control means effects controlled movement of the body from the enabled position to the disabled position.

3. A syringe as claimed in claim 2, wherein the syringe body is controllably movable from the enabled position to a disabling position in which the syringe is positioned for disablement, and then from the disabling position to the disabled position, and the control means effects controlled movement of the body through the disabling position.

4. A syringe as claimed in claim 2, wherein the syringe casing and body are each elongate, and the syringe body is axially and rotatably slideable within the syringe casing, the syringe body sliding from the enabled position to the disabled position.

5. A syringe as claimed in claim 2, wherein the syringe body is axially slidable into the disabled position, sliding of the syringe body into the disabled position retracting the needle into the casing to thereby disable the syringe.

6. A syringe as claimed in claim 2, wherein the control means includes at least one control member on the syringe casing, and at least one control member on the syringe body, the control members on the syringe casing and body inter-engaging during syringe use to thereby effect the controlled movement of the body.

7. A syringe as claimed in claim 6, wherein the control members include at least one control cam and at least one cam follower, the cam and cam follower inter-engaging to effect the controlled movement of the body.

8. A syringe as claimed in claim 7, wherein the cam is located on the body, and the cam follower is located on the casing.

9. A syringe as claimed in claim 7, wherein the cam follower is a follower pin.

10. A syringe as claimed in claim 9, wherein the control cam is elongate and has at least one profiled camming surface extending therealong for operative engagement by the cam follower.

11. A syringe as claimed in claim 10, wherein the control cam includes a camming groove or slot for receiving the cam follower pin therein, the cam follower pin progressively travelling along the camming groove or slot causing relative movement between the syringe body and casing in response to the camming surface profile.

12. A syringe as claimed in claim 7, wherein a single cam follower is provided, the single cam follower being located on an inner surface of the syringe casing and extending generally inwardly therefrom.

13. A syringe as claimed in claim 12, wherein a single control cam is provided, the single control cam being located at an outer surface of the syringe body in facing relation to the single cam follower during engagement therebetween.

14. A syringe as claimed in claim 7, wherein the control cam includes camming stages spaced there along, each with a respective camming surface with which the cam follower successively travels over to cause indexed movement of the syringe body relative to the casing through each camming stage.

15. A syringe as claimed in claim 14, wherein the camming stages including one or more of:
a. a charge draw camming stage during which a liquid charge is drawn into the body chamber;
b. an air ejection camming stage during which air is ejected from the needle;
c. a blood draw camming stage during which blood is drawn from a vein into the needle; and,
d. a charge ejection camming stage during which a liquid charge is ejected from the body chamber,
movement of the plunger during each camming stage forcing the follower into engagement with and to travel over the respective camming surface and thereby cause the syringe body to rotatably slide relative to the syringe casing.

16. A syringe as claimed in claim 15, wherein the camming stages, when provided, are arranged in the order:
a. charge draw camming stage,
b. air ejection camming stage,
c. blood draw camming stage, and
d. charge ejection camming stage along the control cam.

17. A syringe as claimed in claim 14, wherein the control cam includes detent stages adjacent the end of each of the camming stages for ending travel of the cam follower over the preceding camming stage and directing the cam follower toward the successive camming stage.

18. A syringe as claimed in claim 17, wherein each detent stage has a respective camming surface, the camming surfaces in the detent stages being angled relative to the camming surfaces in the preceding camming stages so as to redirect travel of the cam follower and cause the indexed movement of the syringe body through each camming stage.

19. A syringe as claimed in claim 14, wherein the control cam includes a disabling camming stage during which the cam follower and cam disengage and the syringe body moves toward the disabled position.

20. A syringe as claimed in claim 19, wherein the control cam includes a camming groove or slot, and the cam follower is a follower pin received in the camming groove or slot for progressive travel along the camming groove or slot causing relative movement between the syringe body and casing, the camming groove or slot having an open end through which the follower pin moves to exit from the camming groove or slot so as to release the syringe body for longitudinal sliding movement relative to the syringe casing to the disabled position and thereby retract the needle into the casing.

21. A syringe as claimed in claim 19, and including biasing means acting on the syringe body to slidably move the body when in the disabling position to the disabled position.

22. A syringe as claimed in claim 21, wherein the biasing means includes a resilient biasing spring acting between the syringe casing and body, to bias the body along the casing into the disabled position.

23. A syringe as claimed in claim 14, wherein the syringe body is moveable from an enabling position, in which the needle is retracted and housed within the casing, to the enabled position, and the control cam includes an enabling camming stage, movement of the syringe body to the enabled position causing the cam follower to engage the cam and travel over the camming surface of the enabling camming stage, the syringe body sliding along the casing so as to project the needle from the casing for use of the syringe.

24. A syringe as claimed in claim 23, wherein the control cam includes a camming groove or slot, and the cam follower is a follower pin, the camming groove or slot having an open end through which the follower pin moves to enter the camming groove or slot so as to engage the camming surface of the enabling camming stage.

25. A syringe as claimed in claim 11, wherein the camming groove or slot is of generally zig-zag configuration, and the camming and detent stages of the control cam are arranged on opposite sides of apical points of the camming groove or slot.

26. A syringe as claimed in claim 2, wherein the syringe casing includes an access hole through which the needle extends during use of the syringe, the access hole being offset from the needle so that when the needle is retracted into the casing into the disabled position, the retracted needle misaligning with the access hole so as to prevent re-extension of the needle from the casing.

27. A syringe as claimed in claim 2, and including locking means acting on the body to prevent subsequent movement thereof relative to the casing when in the disabled position.

28. A syringe as claimed in claim 27, wherein the locking means includes at least one locking element on each of the syringe body and casing, the locking elements inter-engaging when the syringe body moves into the disabled position, thereby preventing further movement of the syringe body.

29. (canceled)
